(19) United States
(12) Patent Application Publication
(10) Pub. No.: US 2021/0300967 A1
(43) Pub. Date: Sep. 30, 2021

(54) NOVEL PEPTIDES AND ITS DERIVATIVES CAPABLE OF STIMULATING CYTOKINE RELEASE

(71) Applicant: Vanford Bio-Drug Development Limited, Hong Kong (CN)

(72) Inventor: Shing Hing LAU, Hong Kong (CN)

(21) Appl. No.: 17/264,743

(22) PCT Filed: Jul. 31, 2019
(86) PCT No.: PCT/CN2019/098631

§ 371 Date: Jan. 29, 2021

(30) Foreign Application Priority Data
Aug. 1, 2018 (CN) PCT/CN2018/098007

(51) Int. Cl. C07K 7/06; A61P 35/00

(52) U.S. Cl. CPC C07K 7/06; A61K 38/00; A61P 35/00

(57) ABSTRACT

The present invention relates to a novel peptide that can stimulate the release of cytokines, as well as to the use of the peptide as a medicament.

### FIELD OF THE INVENTION

The present invention relates to a novel peptide that can stimulate the release of cytokines, as well as to the use of the peptide as a medicament.

### BACKGROUND OF THE INVENTION

Cytokines play key roles in the regulation of the immune response. These intercellular messengers that are released from innate immune cells allow a coordinated, robust and self-limited response to pathogens and injury. However, over the past two decades there has been a growing interest in the role cytokines can play in cancer immunotherapy.

Cytokines directly stimulate immune effector cells and stromal cells at the tumour site and accordingly, can enhance tumour cell recognition by cytotoxic effector cells. Numerous animal tumour model studies have demonstrated that cytokines have broad anti-tumour activity and this has been translated into a number of cytokine-based approaches for cancer therapy (Lee & Margolin, 2011). Such cytokines display anti-tumour effects both directly, on said cancer cells, or indirectly, through interactions with immune cell populations for example. Many of these mechanisms are yet to be identified despite promising anti-tumour effects in a range of animal models of cancer.

For example, IL-2 was first discovered as a “T-cell growth factor” and has since been approved for the treatment of a number of cancers, including melanoma and renal cell carcinoma, due to its ability to drive T-cell proliferation therefore boosting the anti-tumour immune response (Jiang et al., 2016). In addition to the clinical approval for use in the above cancers, it is well regarded in the literature to be a “go-to” cancer immunotherapy due its ability to activate the immune system, especially in combination with other anticancer immunotherapies (Jiang et al., 2016). Indeed it has been regarded as the “first effective immunotherapy for human cancer” and is likely applicable to all types of cancer (Rosenberg, 2014).

In addition, IL-4 was first described as a “B-cell growth factor”, again boosting the anti-tumour immune response but also directly driving apoptosis in cancer cells including in breast cancer (Nagai and Toi, 2000). The role of IL-4 in tumour immunology is somewhat paradoxical, but it has been shown that IL-4 can induce the most effective immune response among several cytokines in many prophylactic and treatment models of cancer (Li et al., 2009).

IL-12 has often been considered to be one of the most promising candidates for tumour immunotherapy in humans as it can activate both the innate (NK cells) and adaptive (cytotoxic T lymphocytes) arms of the immune response (Lasek et al., 2014). IL-12 based therapies have shown success in a plethora of cancer types including breast, pancreas, cervical, colorectal, lymphoma, melanoma, multiple myeloma, renal, sarcoma and liver (Lasek et al., 2014), and many recent clinical trials have confirmed this efficacy (Lasek & Zagozdzon, 2016).

IFN-γ, also known as type II IFN, can also mediate anti-tumour immunity through a variety of mechanisms including increased activation and survival of immune cells, increased immune effector functions, decreased regulatory T cell immune suppression and increased cytotoxic function (Parker et al., 2016). In addition IFN-γ has been shown to mediate the inhibition of tumour angiogenesis which is a process that drives tumour progression, outgrowth and metastatic spread of all human cancers (Hayakawa et al., 2002).

In light of the above, it is apparent that IL-2, IL-4, IL-12 and IFN-γ are all important cytokines in the development, growth and spread of human cancer, and therefore the modulation of these cytokines is of interest in all types of cancer. There are a number of cytokines in pre-clinical development for cancer immunotherapy. However, delivery of therapeutic cytokines can be problematic, and researchers have been developing alternative strategies including recombinant viral vectors to deliver cytokine genes and PEGylation of cytokine proteins for improved kinetics in the host (Lee and Margolin, 2011). As such, there is a need to find new methods to increase the release of these cytokines, including but not exclusively IL-2, IL-4, IL-12 and IFN-γ, from host cells, and as such promote anti-tumour immunity. The present invention addresses this need.

### SUMMARY OF THE INVENTION

In one aspect of the invention there is provided an isolated polypeptide comprising the amino acid sequence

X1X2X3AX4X5X6X7X8X9X10;

wherein
X1 is selected from PyroQ and A;
X2 is selected from the group consisting of D, E and A;
X3 is selected from the group consisting of T, A and S;
X4 is selected from the group consisting of V, A, I, L and M;
X5 is selected from the group consisting of T and S;
X6 is selected from the group consisting of T, A and S;
X7 is selected from the group consisting of H, K, Q and R;
X8 is selected from the group consisting of E, A, N and Q;
X9 is selected from the group consisting of D, N, Q, and A;
X10 is selected from the group consisting of N, D and A; or a fragment or functional variant thereof.

In one embodiment X1 is PyroQ. In an alternative embodiment, X1 is A. In a second embodiment X2 is E. In a third embodiment X3 is T. In a fourth embodiment X4 is V. In a fifth embodiment X5 is S. In a sixth embodiment X6 is S. In a seventh embodiment X7 is H. In an eighth embodiment X8 is E. In a ninth embodiment X9 is Q. In a tenth embodiment X10 is D.

In one embodiment, there is provided an isolated polypeptide comprising the amino acid sequence defined in any of SEQ ID NOs: 1 to 47 or a fragment or functional variant thereof.

In another aspect of the invention, there is provided an isolated polynucleotide, wherein the isolated polynucleotide comprises
(a) a nucleotide sequence that encodes a polypeptide as defined in any one of SEQ ID NOs 1 to 47 or a variant or fragment thereof, as defined above; or
(b) a nucleotide sequence complementary to (a).

In a further aspect of the invention, there is provided a nucleic acid construct comprising at least one nucleic acid sequence encoding at least one polypeptide as defined in any of SEQ ID NO: 1 to 47 or a functional variant or homolog thereof, wherein preferably at least one said sequence is operably linked to a regulatory sequence. In one embodiment, the regulatory sequence is a constitutive or strong promoter.

In another aspect of the invention there is provided a vector comprising at least one of the polynucleotides described above.

In a further aspect of the invention, there is provided a host cell comprising at least one nucleic acid construct described above.

In another aspect of the invention, there is provided an isolated polypeptide, polynucleotide, nucleic acid construct or host cell as described herein for use as a medicament.

In a further aspect of the invention, there is provided a method of therapy comprising administering at least one isolated polypeptide, polynucleotide or nucleic acid construct described herein to an individual or patient in need thereof.

In another aspect of the invention, there is provided at least one isolated polypeptide, polynucleotide, nucleic acid construct or host cell as described herein for use in the treatment of cancer.

In a further aspect of the invention, there is provided a method of treating cancer, the method comprising administering at least one isolated polypeptide, polynucleotide, nucleic acid construct or host cell described herein to an individual or patient in need thereof.

In one example, the cancer may be selected from one of the following: pancreatic cancer, melanomas, breast cancer, lung cancer, bronchus cancer, colorectal cancer, prostate cancer, pancreas cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, glioma, lymphoma and the like. In one embodiment, the cancer is liver cancer.

In another aspect of the invention there is provided a pharmaceutical composition comprising at least one isolated polypeptide, polynucleotide, nucleic acid construct or host cell as described herein and a pharmaceutically acceptable carrier.

In a further aspect of the invention, there is provided a method of increasing the level of cytokines, the method comprising administering at least one isolated polypeptide, polynucleotide, nucleic acid construct or host cell as described herein to a target cell or patient.

In a final aspect of the invention, there is provided the use of at least one isolated polypeptide, polynucleotide, nucleic acid construct or host cell to increase or stimulate the release of cytokines.

In one embodiment, the cytokine is at least one of IL-2, IL-4, IL-12 and IFN-γ.

### DESCRIPTION OF THE FIGURES

The invention is further described in the following non-limiting figures:

FIG. 1 shows the polypeptides of the invention lack of toxicity against HepG2 (a human liver cancer cell line) cell proliferation when administered at different concentrations.

FIG. 2 shows the effect of administration of the polypeptides on the secretion of cytokines (IL-2, IL-4, IFN-γ and IL-12) from mouse splenic lymphocytes.

FIG. 3 shows the effect of administration of the polypeptide-treated mouse splenic lymphocyte serum (containing cytokines) on HepG2 cell proliferation.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The terms “polypeptide” and “protein” are used interchangeably herein and refer to amino acids in a polymeric form of any length, linked together by peptide bonds.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

We have isolated a number of peptides, which correspond to a modified fragment of rabbit alpha-1-antiproteinase, with the common sequence

X1X2X3AX4X5X6X7X8X9X10;

wherein
X1 is selected from PyroQ and A;
X2 is selected from the group consisting of D, E and A;
X3 is selected from the group consisting of T, A and S;
X4 is selected from the group consisting of V, A, I, L and M;
X5 is selected from the group consisting of T and S;
X6 is selected from the group consisting of T, A and S;
X7 is selected from the group consisting of H, K, Q and R;
X8 is selected from the group consisting of E, A, N and Q;
X9 is selected from the group consisting of D, N, Q, and A;
X10 is selected from the group consisting of N, D and A; or a fragment or functional variant thereof.

that can increase or stimulate the release of cytokines from innate immune cells.

In one embodiment of the invention there is provided an isolated polypeptide comprising the amino acid sequence:

X1X2X3AX4X5X6X7X8X9X10

wherein
X1 is PyroQ;
X2 is selected from the group consisting of D, E and A;
X3 is selected from the group consisting of T, A and S;
X4 is selected from the group consisting of V, A, I, L and M;
X5 is selected from the group consisting of T and S;
X6 is selected from the group consisting of T, A and S;
X7 is selected from the group consisting of H, K, Q and R;
X8 is selected from the group consisting of E, A, N and Q;
X9 is selected from the group consisting of D, N, Q, and A;
X10 is selected from the group consisting of N, D and A;

In one embodiment X2 is E. In a second embodiment X3 is T. In a third embodiment X4 is V. In a fourth embodiment X5 is S. In a fifth embodiment X6 is S. In a sixth embodiment X7 is H. In a seventh embodiment X8 is E. In an eighth embodiment X9 is Q. In a ninth embodiment X10 is D.

In a particularly preferred embodiment, the peptide comprises the sequence PyroQETAVSSHEQD (SEQ ID NO: 1 or SEQ ID NO: 25) or a functional variant thereof. This peptide may be referred to herein as Peptide 1.

In another embodiment, the peptide comprises or consists of a sequence selected from any one of SEQ ID NOs 2 to 24 or SEQ ID NOs 26 to 47.

PyroQ as referred to herein is also known as pyroglutamine. The structure of PyroQ is as follows:

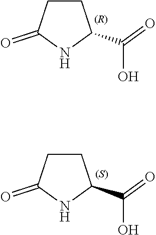

For the avoidance of doubt, PyroQ may also be known as PyroE, PyroGln, PyroGlu or pyroglutamate. Such terms may be used interchangeably herein. PyroQ and PyroE are therefore structurally identical.

The structure of PyroQ when attached to the N-terminus of a peptide is shown below, with the wavy line denoting the point of attachment:

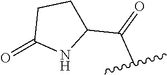 [SMILES: *C(=O)C1CCC(=O)N1]

PyroQ may exist in either the (R) or (S) enantiomer as follows:

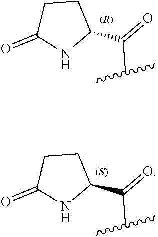

In another aspect of the invention, there is provided an isolated polypeptide comprising an amino acid sequence selected from any one of SEQ ID NOs: 1 to 47 or a fragment or functional variant thereof.

The term “variant” or “functional variant” as used herein with reference to any of SEQ ID NOs: 1 to 47 refers to a variant sequence or part of the sequence which retains the biological function of the full non-variant sequence. A functional variant also comprises a variant, which has sequence alterations that do not affect function, for example in non-conserved residues. Also encompassed is a variant that is substantially identical, i.e. has only some sequence variations and is biologically active. Alterations in a nucleic acid or amino acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

The polypeptides of the invention may include additional amino acids, such as N-terminal additions useful in the purification of the polypeptide, such as, for example, binding tags and cleavage recognition sites.

In one example, the binding tag binds glutathione; the tag may be glutathione S-transferase (GST). In another example, the tag may be biotin. The binding tag target is preferably immobilised on a solid support; this allows the bound polypeptide to be easily isolated from unbound product. Other suitable binding tags immobilised on similar solid supports could be used.

In another example, the cleavage recognition site comprises a sequence that recognises thrombin, enterokinase, or factor Xa, among others. Preferably this site is within or adjacent the binding tag.

The polypeptides of the invention may also be modified. Examples of modifications include any post-translational modifications such as, but not limited to glycosylation, alkylation (for example, methylation), acetylation, amidation, hydroxylation, ubiquitination, sulfation and phosphorylation, any chemical modification or any modification comprising a non-covalent and covalent linkage to anther protein or peptide.

A further aspect of the present invention provides a method of producing or purifying such polypeptides, the method comprising expressing a vector comprising a nucleotide sequence encoding any of SEQ ID NOs 1 to 47 in a host cell, wherein the vector preferably comprises a regulatory sequence as described herein, the vector additionally preferably comprising a nucleotide sequence encoding a binding tag; allowing the expressed polypeptide to bind to the target of said binding tag; and causing said bound polypeptide to be released from said target. The host cell may be eukaryotic, for example, a mammal, other vertebrate or invertebrate, insect, fungal, or plant cell; or may be prokaryotic, for example, bacterial; and may use vectors of bacterial, yeast, other eukaryotic, other non-eukaryotic, or virus sequence origin. The method may then comprise the step of cleaving the polypeptide at the recognition site.

The polypeptide may be produced or synthesised by any method known to the skilled person, for example, in one embodiment, the polypeptide is produced using chemical synthesis. One example of a method to synthesise the polypeptides of the invention is provided in Example 2.

In another aspect of the invention, there is provided an isolated polynucleotide, wherein the isolated polynucleotide comprises
(a) a nucleotide sequence that encodes a polypeptide selected from any one of SEQ ID NOs 1 to 47 or a variant or fragment thereof, or
(b) a nucleotide sequence complementary to (a);
(c) a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98%, or at least 99% overall sequence identity to either (a) or (b); or
(d) a nucleic acid sequence encoding a polypeptide selected from any one of SEQ ID NOs 1 to 47 that is capable of hybridising under stringent conditions as defined herein to the nucleic acid sequence of any of (a) to (c).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further aspect of the invention, there is provided a nucleic acid construct or vector comprising a nucleic acid sequence encoding a polypeptide selected from any one of SEQ ID NO: 1 to 47 or a functional variant or homolog thereof, wherein preferably said sequence is operably linked to a regulatory sequence. The regulatory sequence may be any form or promoter, such as a constitutive, strong, regulated or inducible promoter that leads to expression of the nucleic acid when expressed in a target or host cell. Examples include, but are not limited to the viral promoters cytomegalovirus (CMV) promoter and SV40 (simian vacuolating virus 40) and non-viral promoters such elongation factor (EF)-1 and actin.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

The term “operably linked” as used herein refers to a functional linkage between the promoter sequence and the gene of interest, such that the promoter sequence is able to initiate transcription of the gene of interest.

In another aspect of the invention there is provided a vector comprising the polynucleotide described above. Vectors may include bacterial or yeast plasmids, cosmids, bacteriophages, artificial chromosomes or plant or mammalian viruses. Preferably the vector is an expression vector, also known as an expression construct. In one embodiment, the expression vector may comprise an origin or replication, at least one selectable marker and a multiple cloning site suitable for the insertion of the nucleic acid sequence to be expressed. Expression vectors can be produced by any one of numerous techniques known to a person skilled in the art.

In a further aspect of the invention, there is provided a host cell comprising the nucleic acid construct. The host cell may be prokaryotic or eukaryotic, and may include bacterial cells, fungal cells such as yeast, plant cells, insect cells, or mammalian cells. As on example only, the mammalian host cell may be selected from CHO (Chinese hamster ovary) cells, COS, HEK or HeLa. Alternatively, the host cell may be an immune cell, such as a lymphocyte (B lymphocyte or T lymphocyte), macrophage or mast cell, or a liver or spleen cell, endothelial cell, fibroblast, or stromal cell. Also provided is a host cell comprising an exogenous polynucleotide according to the above aspects of the invention. Preferably the host cell expresses said polynucleotide.

In another aspect of the invention, there is provided a method of producing a polypeptide as described herein, the method comprising introducing and expressing a nucleic acid construct as described into a host cell and isolating the polypeptide.

The nucleic acid construct is introduced into said host cell through a process called transformation or transfection. The term “introduction” or “transformation” or “transfection” as referred to herein encompasses the transfer of an exogenous polynucleotide into a host cell, irrespective of the method used for transfer. The polynucleotide may be transiently or stably introduced into a host cell and may be maintained non-integrated, for example, as a plasmid. Alternatively, it may be integrated into the host genome.

Transformation is now a routine technique in many species. Advantageously, any of several transformation methods may be used to introduce the gene of interest into a suitable ancestor cell. Transformation methods include the use of liposomes, electroporation, chemicals that increase free DNA uptake, injection of the DNA directly into the host cell, particle gun bombardment, transformation using viruses or pollen and microprojection.

In another aspect of the invention, there is provided a method of increasing the level of cytokines, the method comprising administering at least one or any combination of isolated polypeptide or polynucleotide or nucleic acid construct as described herein to a target cell or patient in need thereof.

In one embodiment, the cytokine is at least one of IL-2, IL-4, IL-12 and IFN-γ.

In a further embodiment, the release of cytokine is increased by between 10 and 200%, more preferably between 10 and 150% compared to the level in control cells. In one embodiment, control cells are unstimulated (i.e. no peptide administered) cells.

In one embodiment, the level of IL-2 is increased by between 5 and 150%, more preferably by at least 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150% or more compared to the level in a control. In a specific embodiment, the peptide is SEQ ID NO. 1 or SEQ ID NO. 25 and the level of increase is between 5 and 40%, more preferably between 5 and 15%.

In another embodiment, the level of IL-4 is increased by between 5 and 100%, more preferably by at least 5%, 10%, 15%, 20%, 25%, 30%, 35%, 40%, 45%, 50%, 55%, 60%, 65%, 70%, 75%, 80%, 85%, 90%, 95%, 100% or more compared to the level in a control. In a specific embodiment, the peptide is SEQ ID NO. 1 or SEQ ID NO. 25 and the level of increase is between 5 and 40%, more preferably between 10 and 20% compared to the level in a control.

In a further embodiment, the level of IFN-γ is increased by between 2 and 100%, more preferably by at least 2%, 5%, 10%, 20%, 25%, 30%, 35%, 40%, 45%, 50%, 55%, 60% 65%, 70%, 75%, 80%, 85%, 90%, 95%, 100% or more compared to the level in a control. In a specific embodiment, the peptide is SEQ ID NO. 1 or SEQ ID NO. 25 and the level of increase is between 25 and 60%, more preferably between 30 and 40% compared to the level in a control.

In another embodiment, the level of IL-12 is increased by between 10 and 400%, more preferably by at least 10%, 50%, 100%, 150%, 200%, 250%, 300%, 350%, 400% or more compared to the level in a control. In a specific embodiment, the peptide is SEQ ID No. 1 or SEQ ID NO. 25 and the level of increase is between 150 and 250%, more preferably between 180 and 220% compared to the level in a control.

In another aspect of the invention, there is provided at least one or any combination of isolated polypeptide or polynucleotide as described herein for use as a medicament.

In a further aspect of the invention, there is provided a method of therapy comprising administering at least one or any combination of isolated polypeptide or polynucleotide or nucleic acid construct described herein to an individual or patient in need thereof.

In another aspect of the invention, there is provided at least one or any combination of isolated polypeptide, polynucleotide or nucleic acid construct as described herein for use in the treatment of cancer.

In a further aspect of the invention, there is provided a method of treating cancer, the method comprising administering at least one or any combination of isolated polypeptide, polynucleotide or nucleic acid construct as described herein to an patient in need thereof.

In another aspect of the invention, there is provided the use of at least one or any combination of isolated polypeptide or polynucleotide or nucleic acid construct as described herein in the preparation of a medicament for the treatment of cancer.

In one example, the cancer may be selected from one of the following: pancreatic cancer, melanomas, breast cancer, lung cancer, bronchus cancer, colorectal cancer, prostate cancer, pancreas cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, glioma, lymphoma and the like. In one embodiment, the cancer is liver cancer.

In another aspect of the invention, there is provided a method of decreasing tumour cell proliferation the method comprising administering at least one isolated polypeptide or polynucleotide or nucleic acid construct as described herein to a target cell or patient in need thereof. In one embodiment, the level of decrease is between 10 and 60%, more preferably by at least 10%, 15%, 20%, 25%, 30%, 35%, 40%, 45%, 50%, 55%, 60% or more compared to the level in a control. In a specific embodiment, the peptide is SEQ ID NO. 1 or SEQ ID NO. 25 and the level of decrease is between 5 and 20%, more preferably between 10 and 15% compared to the level of control.

As used herein, a control may be an individual, patient or cell that has not been treated with at least one polypeptide of the invention.

In another aspect of the invention, there is provided a pharmaceutical composition comprising any one or at least one of the peptides, polynucleotides, vectors or constructs described herein and a pharmaceutically acceptable carrier. The composition will typically be formulated using well-known methods prior to administration into a patient.

Administration of the polypeptides or pharmaceutical compositions of the invention may be accomplished orally or parenterally. Methods of parenteral delivery include topical, intra-arterial, intramuscular, subcutaneous, intramedullary, intrathecal, intraventricular, intravenous, intraperitoneal, mucosal or intranasal administration. In addition to the active ingredients, such compositions may comprise suitable pharmaceutically acceptable carriers comprising excipients and other components which facilitate processing of the active compounds into preparations suitable for pharmaceutical administration.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical preparations for oral use can be obtained through combination of active compounds with a solid excipient, optionally grinding a resulting mixture, and processing the mixture of granules, after adding suitable additional compounds if desired to obtain tablets or dragee cores. Suitable excipients include carbohydrate or protein fillers such as sugars, including lactose, sucrose, mannitol, sorbitol; starch from corn, wheat, rice, potato, or other plants; cellulose such as methylcellulose, hydroxypropylmethylcellulose, or sodium carboxymethylcellulose; and gums including arabic and tragacanth; as well as proteins such as gelatin and collagen. If desired, disintegrating or solubilising agents may be added, such as cross linked polyvinyl pyrrolidone, agar, alginic acid, or a salt thereof.

Dragee cores can be provided with suitable coatings such as concentrated sugar solutions, which may also contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for product identification or to characterise the quantity of active compound.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally stabilisers. In soft capsules, the active compounds can be dissolved or suspended in suitable liquids, such as fatty oils, liquid paraffin, or liquid polyethylene glycol with or without stabilisers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions. Pharmaceutical compositions may also include adjuvants to enhance or modulate antigenicity.

For topical or nasal administration, penetrants appropriate to the particular barrier to be permeated may be used in the formulation.

In a further aspect of the invention, there is provided the use of an isolated polypeptide, polynucleotide, nucleic acid construct or host cells described herein as an adjuvant. In one embodiment, the polypeptide, polynucleotide or nucleic acid construct may be used as a cellular adjuvant or immunotherapeutic to chemoattract cells to mediate innate or adaptive immunity and increase antigenicity. In one embodiment, the polypeptide, polynucleotide or nucleic acid construct of the invention is co-administered with an antigen or another immunotherapeutic.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“cited documents”) and all documents cited or referenced in the cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### Example 1

Experiment Outline

We have assessed the peptides' anti-tumour activity by the enhancement of the immune system, with three separate experiments, as follows:

1.) Their ability to prohibit the proliferation of human tumour cells is assessed.

2.) Their ability to increase the secretion of immune-cell related cytokines upon treatment with mouse splenic lymphocytes.

3.) Lastly, the serum of mouse splenic lymphocytes after treatment with peptides were isolated and added directly to human tumour cells, where the inhibitory effect on tumour cell proliferation is assessed.

Experimental

1—Peptide's Cytotoxicity on Tumour Cells

a) Cell culture—HepG2 cells were grown in DMEM high glucose medium (FBS containing 10% by volume) and cultured in a 5% CO₂ incubator at 37° C. Trypsin digestion, conventional cell passage.

b) CCK-8 detection of cell proliferation—HepG2 cells in logarithmic growth phase were made into a cell suspension, and 5×10³ cells were seeded in each well of a 96-well flat bottom plate. After overnight culture, the cells were grown in adherent culture, and replaced with medium containing different concentrations of polypeptides dissolved in PBS/DMSO. Blank solvents were used as a negative control. Each sample is performed in triplicates and incubated for 15 h. Afterwards, cck-8 (10 μl/well) was added and incubated for 1 h. The absorbance A value at wavelength 450 nm was measured on enzyme-linked immunosorbent.

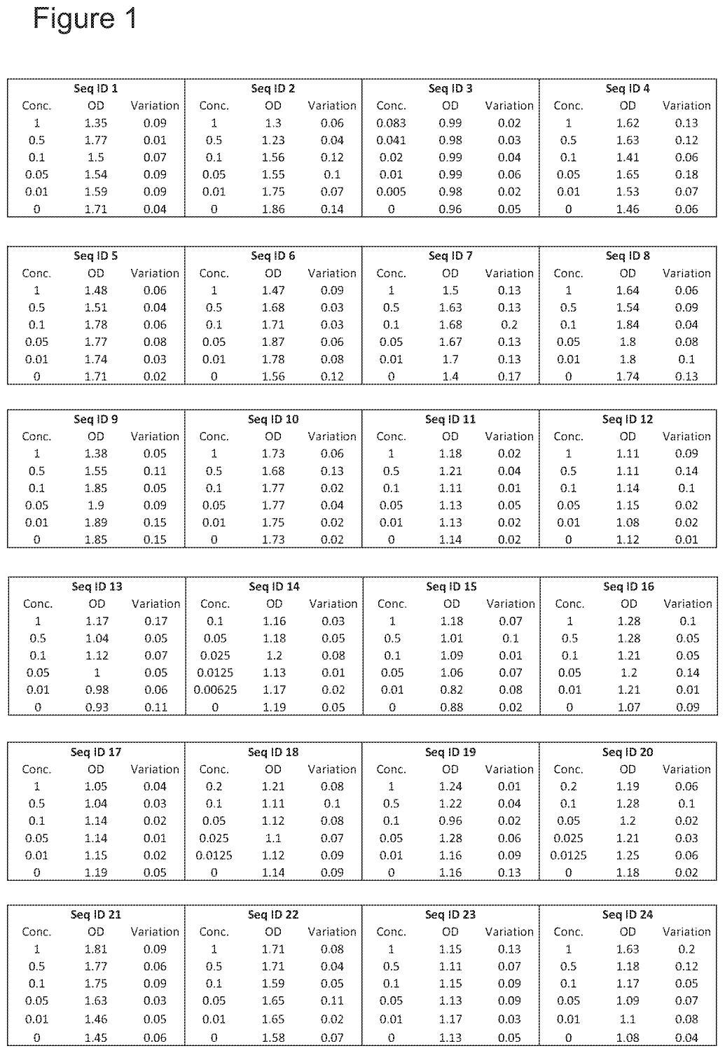

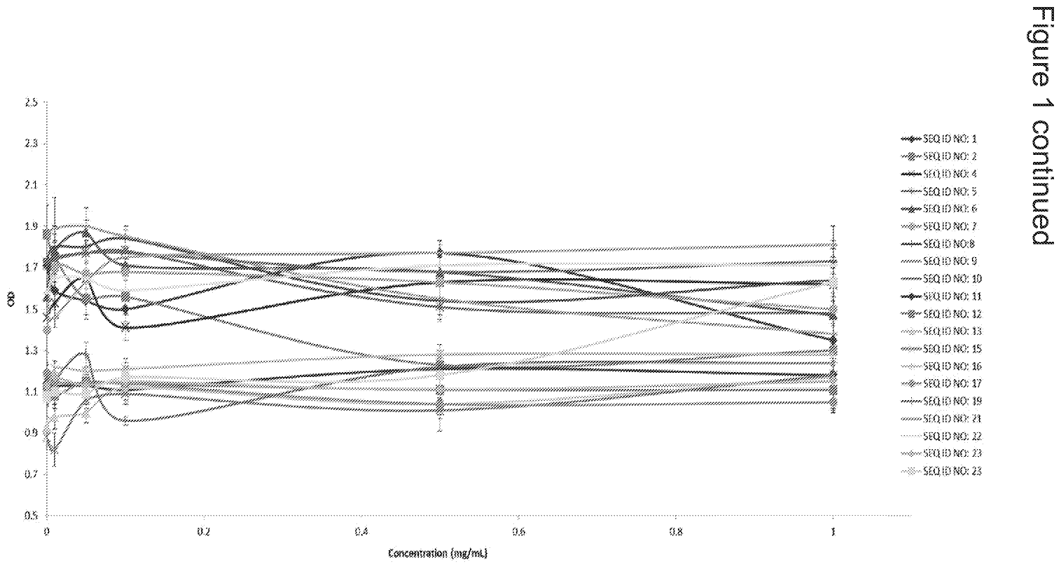

The result of this experiment is shown in FIG. 1. As shown in FIG. 1, the peptides per se have no effect on tumour cell proliferation and therefore are not cytotoxic per se.

2. Study on the Immunological Activity of Candidate Peptides

Mouse splenic lymphocytes were prepared and tested with the candidate peptides for immunological activity by stimulation experiments.

a) Male Kunming (KM) mouse were killed by cervical dislocation and the spleen was aseptically dissected. The spleen was then placed in a dish with 5 mL lymphocyte separation fluid and grinded. The cells were collected by centrifugation at 800 g for 30 min, and the supernatant was removed. The cells were washed with 10 mL of RPMI 1640 cell culture medium (1640) and isolated by centrifugation at 250 g for 10 min. 1640 and Fetal Bovine Serum (FBS) were used to resuspend the cells and the concentration of lymphocytes were adjusted to 5×10⁶/mL. Each of the wells in the 96-well plate was charged with 100 μL of suspended cells, and different concentrations of candidate polypeptides (dissolved in either PBS/DMSO) were added in triplicates and incubated for 24 h. ConA/LPS were used as positive control.

b) ELISA—After incubation, the samples were centrifuged, supernatant isolated and tested according to ELISA kit instructions for cytokine concentration detection. Briefly, to each well, 50 μL RD-14 was added, and then 50 μL of sample (standard, controls and isolated supernatant from step A). After mixing, the solution was incubated for 2 h at room temperature. The samples were washed 5 times; 100 μL of binding fluid was added and incubated for 2 h at room temperature. The samples were then washed 5 times, and 100 μL of substrate solution was added, incubated in the dark for 30 min at room temperature, and then 100 mL of quenching solution was added, and the subsequent OD value was measured (450 nm).

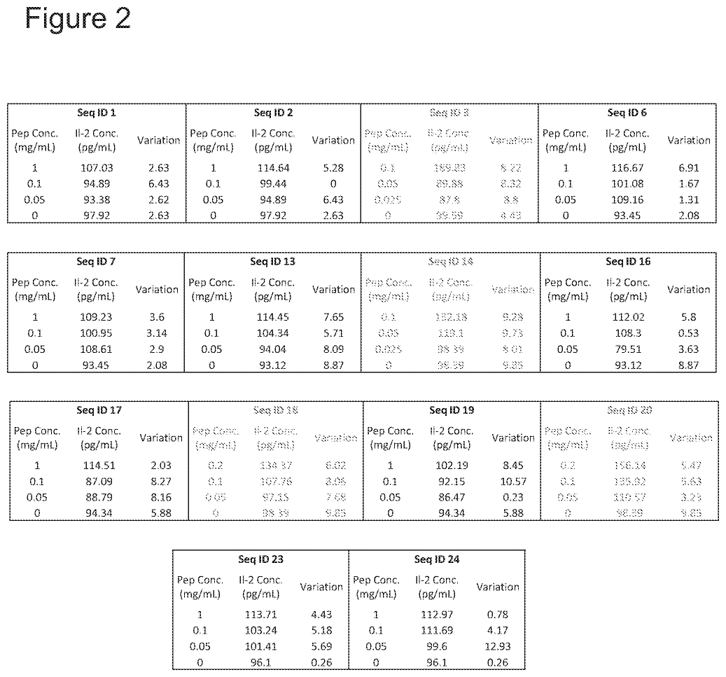

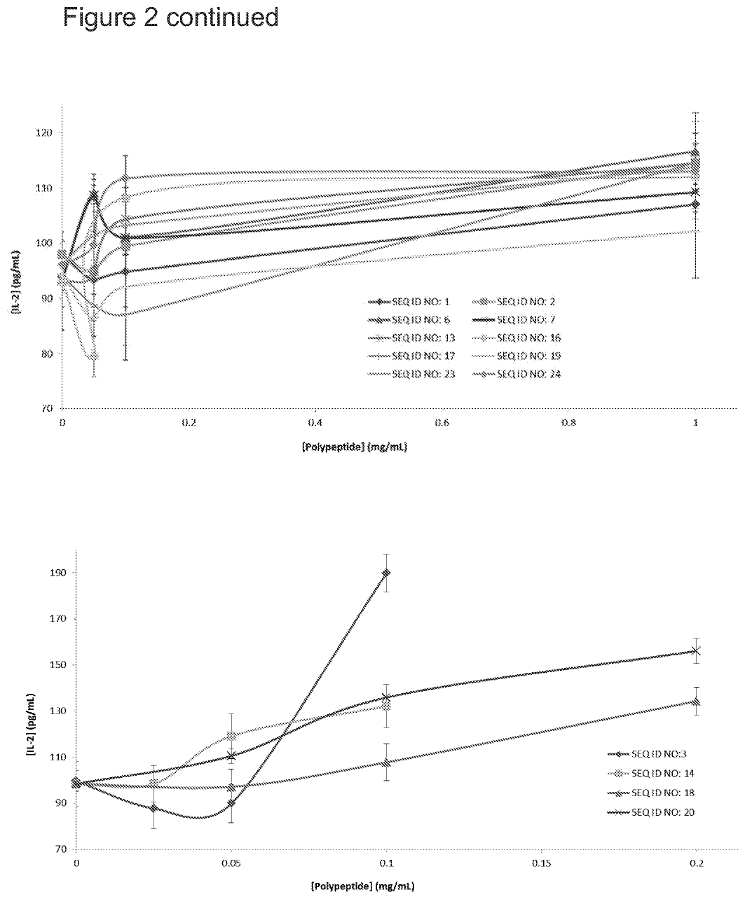

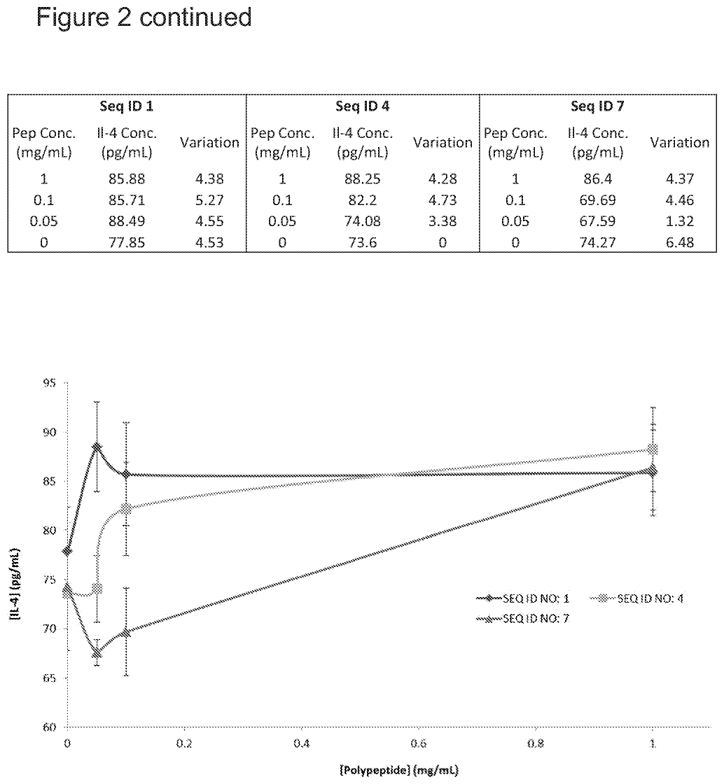

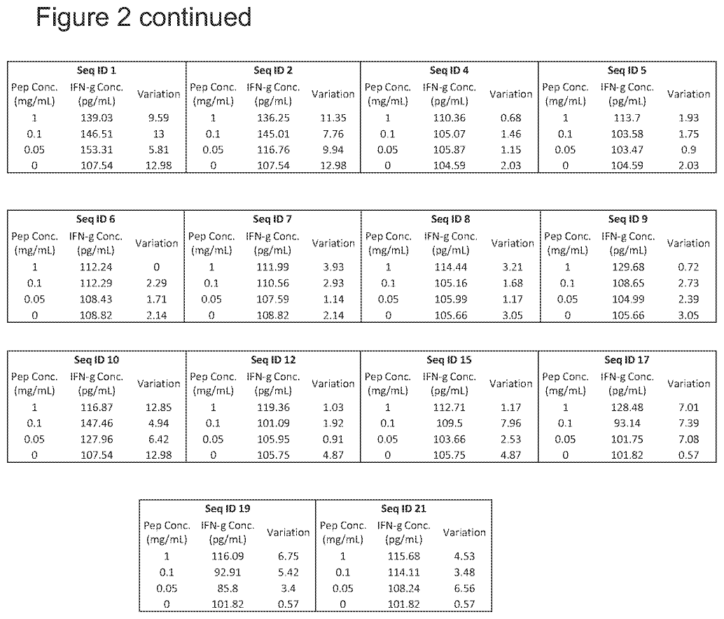

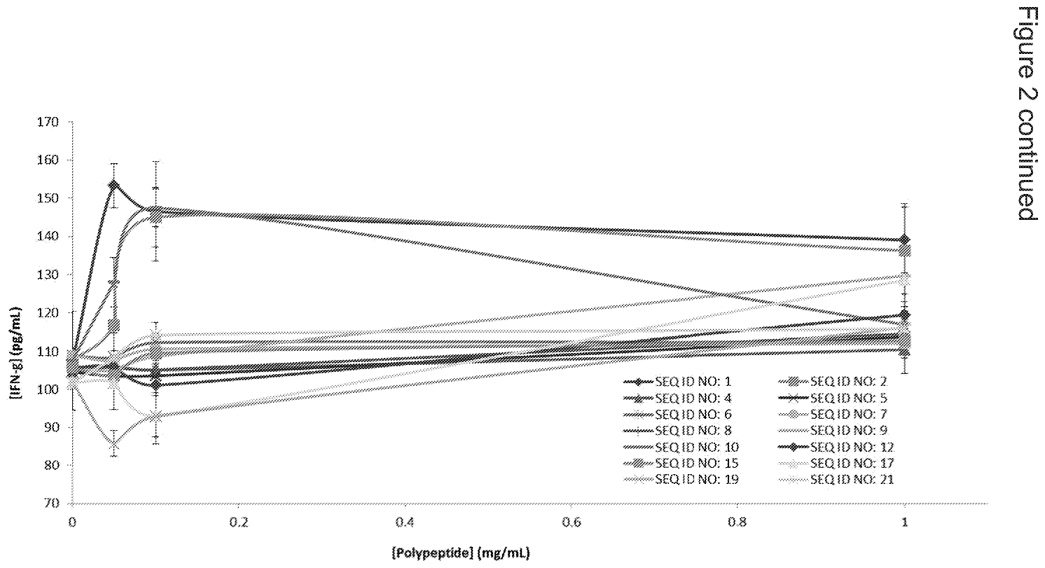

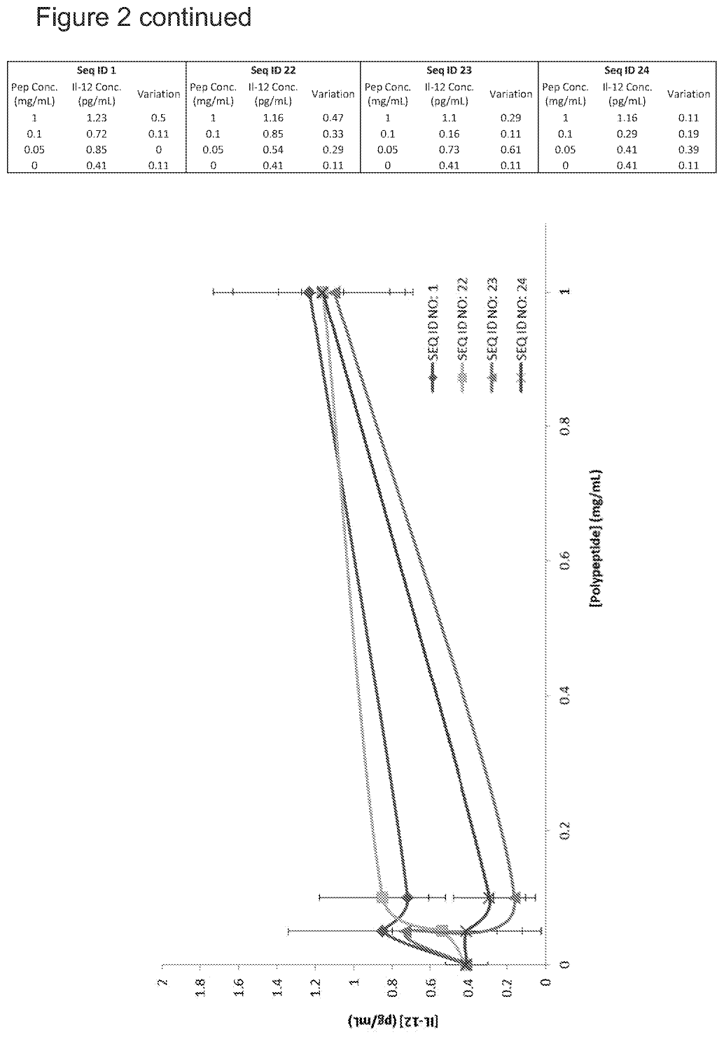

The result of this experiment is shown in FIG. 2. As shown in FIG. 2, increasing concentrations of the polypeptides led to a respective increase in the amount of IL-2, IL-4, IFN-γ and IL-12.

3. The Immune Activity of Candidate Peptides on Tumour Cells

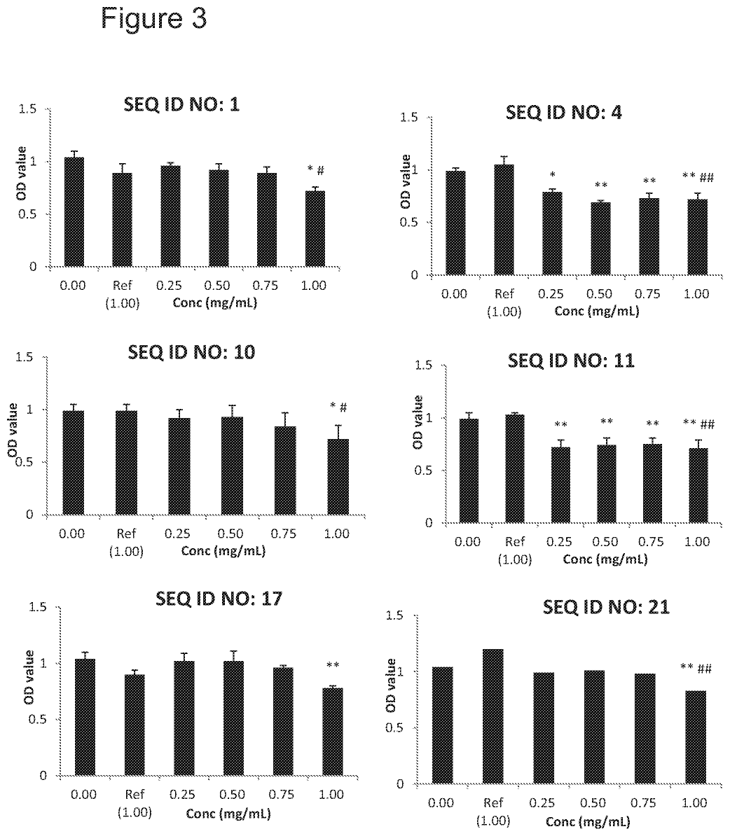

Spleen lymphocytes are stimulated according to steps (2a), and the supernatant containing the immune cytokine is obtained by centrifugation. The supernatant is added to overnight cultured HepG2 cells and cultured for an additional 24 h. Two negative controls were performed, using (1) blank solvent and (2) polypeptides dissolved in PBS/DMSO. The cell viability was detected by MTT assay and the effect on tumour cell proliferation was analysed. The results of this experiment are shown in FIG. 3. “0.00” concentration as shown in the Figures represents spleen supernatum that has not been challenged with peptides. Ref “(1.00)” refers to peptides at a concentration of 1 mg/mL directly administered to HepG2 cells, therefore not containing any cytokines (similar to Experiment 1, and shown in FIG. 1). As shown in FIG. 3, spleen supernatum challenged with various concentrations of the presently claimed peptides significantly decreased cell proliferation in model human liver carcinoma cells (HepG2 cells).

### Example 2

Exemplary Method for the Production of the Peptides

In one example, the peptides of the invention can be produced using the following method:

a) The manual peptide reactor is charged with 200 mg of 2-chlorotrityl resin (1.0 mmol/g) and swelled with DCM for 30 min, and then run dry. The corresponding Fmoc-protected amino acid (0.4 mmol, 2 eq), DIEA (0.4 mmol, 2 eq), and suitable amount of DMF and DCM was mixed by bubbling N₂ for 1 h. MeOH (1.0 mmol, 5 eq) and DIEA (0.4 mmol, 2 eq) was then added and reacted for 30 min to cap unreacted sites.

b) The resin was washed with DMF (×5) and then reacted with 20% piperidine in DMF for 10 min to remove Fmoc group. Repeat the addition of 20% piperidine in DMF twice. The resultant resin was washed with DMF and then checked with chloranil test.

c) The next Fmoc amino acid (0.4 mmol, 2 eq), HBTU (0.4 mmol, 2 eq) and DIEA (0.4 mmol, 2 eq) in DMF/DCM was added to the resin and mixed by bubbling N₂ for 1 h, and the resins were checked by chloranil test. If coupling is incomplete, add another portion of Fmoc amino acid and coupling reagents and react for a further 1 h.

d) Repeat steps (b) and (c) until the sequence is complete.

e) After the last Fmoc group is removed, transfer the resin to a separate glass vial and then add cleavage cocktail (95% TFA, 2% EDT, 2% TIS and 1% water) at room temperature and leave for 3 h. The resultant mixture is filtered, concentrated, and transferred to cold diethyl ether (10× volume of concentrated mixture). Peptide is isolated by centrifugation. The subsequent pellet is dried in vacuo and then purified by HPLC.

### REFERENCES

1. Lee & Margolin, Cancers, 2011, 3, 3856-93.
2. Jiang, Zhou & Ren, Oncoimmunology, 2016, 5, e1163462.
3. Nagai & Toi, Breast Cancer, 2000, 7, 181-6.
4. Parker, Rautela & Hertzog, Nat. Rev. Cancer, 2016, 16, 131-44.
5. Lasek et al., Cancer Immunol. Immunother., 2014, 63, 419-35.
6. Rosenberg, J Immunol., 2014, 192, 5451-8.
7. Li, Chen & Qin. Cell Mol. Immunol., 2009, 6, 415-22.
8. Lasek & Zagozdzon (2016) Clinical Trials with IL-12 in Cancer Immunotherapy. In: Interleukin 12: Antitumor Activity and Immunotherapeutic Potential in Oncology. SpringerBriefs in Immunology. Springer, Cham.
9. Hayakawa et al., Blood, 2002, 100, 1728-33.

SEQUENCE LISTING

SEQ ID NO: 1 = PyroQ-ETAVSSHEQD
SEQ ID NO: 2 = AETAVSSHEQD
SEQ ID NO: 3 = PyroQ-ATAVSSHEQD
SEQ ID NO: 4 = PyroQ-EAAVSSHEQD
SEQ ID NO: 5 = PyroQ-ETAASSHEQD
SEQ ID NO: 6 = PyroQ-ETAVSAHEQD (7)
SEQ ID NO: 7 = PyroQ-ETAVSSHAQD (9)
SEQ ID NO: 8 = PyroQ-ETAVSSHEAD (10)
SEQ ID NO: 9 = PyroQ-ETAVSSHEQA (11)
SEQ ID NO: 10 = PyroQ-DTAVSSHEQD (12)
SEQ ID NO: 11 = PyroQ-ESAVSSHEQD (15)
SEQ ID NO: 12 = PyroQ-ETAISSHEQD (20)
SEQ ID NO: 13 = PyroQ-ETALSSHEQD (21)
SEQ ID NO: 14 = PyroQ-ETAMSSHEQD (22)
SEQ ID NO: 15 = PyroQ-ETAVTSHEQD (23)
SEQ ID NO: 16 = PyroQ-ETAVSTHEQD (24)
SEQ ID NO: 17 = PyroQ-ETAVSSKEQD (25)
SEQ ID NO: 18 = PyroQ-ETAVSSQEQD (27)
SEQ ID NO: 19 = PyroQ-ETAVSSREQD (28)
SEQ ID NO: 20 = PyroQ-ETAVSSHNQD (30)
SEQ ID NO: 21 = PyroQ-ETAVSSHQQD (31)
SEQ ID NO: 22 = PyroQ-ETAVSSHEDD (32)
SEQ ID NO: 23 = PyroQ-ETAVSSHEND (34)
SEQ ID NO: 24 = PyroQ-ETAVSSHEQN (36)
SEQ ID NO: 25 = PyroE-ETAVSSHEQD
SEQ ID NO: 26 = PyroE-ATAVSSHEQD
SEQ ID NO: 27 = PyroE-EAAVSSHEQD
SEQ ID NO: 28 = PyroE-ETAASSHEQD
SEQ ID NO: 29 = PyroE-ETAVSAHEQD (7)
SEQ ID NO: 30 = PyroE-ETAVSSHAQD (9)
SEQ ID NO: 31 = PyroE-ETAVSSHEAD (10)
SEQ ID NO: 32 = PyroE-ETAVSSHEQA (11)
SEQ ID NO: 33 = PyroE-DTAVSSHEQD (12)
SEQ ID NO: 34 = PyroE-ESAVSSHEQD (15)
SEQ ID NO: 35 = PyroE-ETAISSHEQD (20)
SEQ ID NO: 36 = PyroE-ETALSSHEQD (21)
SEQ ID NO: 37 = PyroE-ETAMSSHEQD (22)
SEQ ID NO: 38 = PyroE-ETAVTSHEQD (23)
SEQ ID NO: 39 = PyroE-ETAVSTHEQD (24)
SEQ ID NO: 40 = PyroE-ETAVSSKEQD (25)
SEQ ID NO: 41 = PyroE-ETAVSSQEQD (27)
SEQ ID NO: 42 = PyroE-ETAVSSREQD (28)
SEQ ID NO: 43 = PyroE-ETAVSSHNQD (30)
SEQ ID NO: 44 = PyroE-ETAVSSHQQD (31)
SEQ ID NO: 45 = PyroE-ETAVSSHEDD (32)
SEQ ID NO: 46 = PyroE-ETAVSSHEND (34)
SEQ ID NO: 47 = PyroE-ETAVSSHEQN (36)

1. An isolated polypeptide comprising the amino acid sequence
X1X2X3AX4X5X6X7X8X9X10;
wherein
X1 is selected from PyroQ and A;
X2 is selected from the group consisting of D, E and A;
X3 is selected from the group consisting of T, A and S;
X4 is selected from the group consisting of V, A, I, L and M;
X5 is selected from the group consisting of T and S;
X6 is selected from the group consisting of T, A and S;
X7 is selected from the group consisting of H, K, Q and R;
X8 is selected from the group consisting of E, A, N and Q;
X9 is selected from the group consisting of D, N, Q, and A;
X10 is selected from the group consisting of N, D and A;
or a fragment of functional variant thereof.

2. The isolated polypeptide of claim 1, wherein X1 is PyroQ, X2, is E, or X1 is PyroQ and X2 is E.

3. (canceled)

4. The isolated polypeptide of claim 1, wherein X3 is T, X4 is V, or X3 is T and X4 is V.

5. (canceled)

6. The isolated polypeptide of claim 1, wherein X5 is S.

7. The isolated polypeptide of claim 1, wherein X6 is S

8. The isolated polypeptide of claim 1, wherein X7 is H.

9. The isolated polypeptide of claim 1, wherein X8 is E.

10. The isolated polypeptide of claim 1, wherein X9 is Q.

11. The isolated polypeptide of claim 1, wherein X10 is D.

12. The isolated polypeptide of claim 1, comprising an amino acid sequence selected from the group consisting of SEQ ID NO: 1 to 47 or a fragment or functional variant thereof.

13. An isolated polynucleotide, wherein the polynucleotide encodes the polypeptide of claim 1.

14. The isolated polynucleotide of claim 13, wherein the polynucleotide encodes a polypeptide selected from any one of SEQ ID NOs 1 to 47.

15. A nucleic acid construct comprising at least one isolated polynucleotide of claim 13.

16. A vector comprising the nucleic acid construct of claim 15.

17. A host cell comprising at least one vector of claim 16.

18. (canceled)

19. A method of therapy comprising administering at least one isolated polypeptide of claim 1 to a patient in need thereof.

20. A method of increasing the level or stimulating the release of cytokines, the method comprising administering at least one isolated polypeptide of claim 1 to a target cell or patient.

21. The method of claim 20, wherein the cytokine is at least one of IL-2, IL-4, IFN-γ and IL-12.

22. (canceled)

23. A method of treating cancer, the method comprising administering at least one isolated polypeptide of claim 1 to a patient in need thereof.

24. A pharmaceutical composition comprising the isolated polypeptide of claim 1 and a pharmaceutically acceptable carrier.


SEQUENCE LISTING (47 sequences)

SEQ ID NO: 1 - protein, 11 aa, Artificial Sequence
  PyroQ peptide
  PyroQ (1)..(1)
QETAVSSHEQD

SEQ ID NO: 2 - protein, 11 aa, Artificial Sequence
  Peptide
AETAVSSHEQD

SEQ ID NO: 3 - protein, 11 aa, Artificial Sequence
  PyroQ peptide
  PyroQ (1)..(1)
QATAVSSHEQD

SEQ ID NO: 4 - protein, 11 aa, Artificial Sequence
  PyroQ peptide
  PyroQ (1)..(1)
QEAAVSSHEQD

SEQ ID NO: 5 - protein, 11 aa, Artificial Sequence
  PyroQ peptide
  PyroQ (1)..(1)
QETAASSHEQD

SEQ ID NO: 6 - protein, 11 aa, Artificial Sequence
  PyroQ peptide
  PyroQ (1)..(1)
QETAVSAHEQD

SEQ ID NO: 7 - protein, 11 aa, Artificial Sequence
  PyroQ peptide
  PyroQ (1)..(1)
QETAVSSHAQD

SEQ ID NO: 8 - protein, 11 aa, Artificial Sequence
  PyroQ peptide
  PyroQ (1)..(1)
QETAVSSHEAD

SEQ ID NO: 9 - protein, 11 aa, Artificial Sequence
  PyroQ peptide
  PyroQ (1)..(1)
QETAVSSHEQA

SEQ ID NO: 10 - protein, 11 aa, Artificial Sequence
  PyroQ peptide
  PyroQ (1)..(1)
QDTAVSSHEQD

SEQ ID NO: 11 - protein, 11 aa, Artificial Sequence
  PyroQ peptide
  PyroQ (1)..(1)
QESAVSSHEQD

SEQ ID NO: 12 - protein, 11 aa, Artificial Sequence
  PyroQ peptide
  PyroQ (1)..(1)
QETAISSHEQD

SEQ ID NO: 13 - protein, 11 aa, Artificial Sequence
  PyroQ peptide
  PyroQ (1)..(1)
QETALSSHEQD

SEQ ID NO: 14 - protein, 11 aa, Artificial Sequence
  PyroQ peptide
  PyroQ (1)..(1)
QETAMSSHEQD

SEQ ID NO: 15 - protein, 11 aa, Artificial Sequence
  PyroQ peptide
  PyroQ (1)..(1)
QETAVTSHEQD

SEQ ID NO: 16 - protein, 11 aa, Artificial Sequence
  PyroQ peptide
  PyroQ (1)..(1)
QETAVSTHEQD

SEQ ID NO: 17 - protein, 11 aa, Artificial Sequence
  PyroQ peptide
  PyroQ (1)..(1)
QETAVSSKEQD

SEQ ID NO: 18 - protein, 11 aa, Artificial Sequence
  PyroQ peptide
  PyroQ (1)..(1)
QETAVSSQEQD

SEQ ID NO: 19 - protein, 11 aa, Artificial Sequence
  PyroQ peptide
  PyroQ (1)..(1)
QETAVSSREQD

SEQ ID NO: 20 - protein, 11 aa, Artificial Sequence
  PyroQ peptide
  PyroQ (1)..(1)
QETAVSSHNQD

SEQ ID NO: 21 - protein, 11 aa, Artificial Sequence
  PyroQ peptide
  PyroQ (1)..(1)
QETAVSSHQQD

SEQ ID NO: 22 - protein, 11 aa, Artificial Sequence
  PyroQ peptide
  PyroQ (1)..(1)
QETAVSSHEDD

SEQ ID NO: 23 - protein, 11 aa, Artificial Sequence
  PyroQ peptide
  PyroQ (1)..(1)
QETAVSSHEND

SEQ ID NO: 24 - protein, 11 aa, Artificial Sequence
  PyroQ peptide
  PyroQ (1)..(1)
QETAVSSHEQN

SEQ ID NO: 25 - protein, 10 aa, Artificial Sequence
  PyroE peptide
  PyroE (1)..(1)
ETAVSSHEQD

SEQ ID NO: 26 - protein, 10 aa, Artificial Sequence
  PyroE peptide
  PyroE (1)..(1)
ATAVSSHEQD

SEQ ID NO: 27 - protein, 10 aa, Artificial Sequence
  PyroE peptide
  PyroE (1)..(1)
EAAVSSHEQD

SEQ ID NO: 28 - protein, 10 aa, Artificial Sequence
  PyroE peptide
  PyroE (1)..(1)
ETAASSHEQD

SEQ ID NO: 29 - protein, 10 aa, Artificial Sequence
  PyroE peptide
  PyroE (1)..(1)
ETAVSAHEQD

SEQ ID NO: 30 - protein, 10 aa, Artificial Sequence
  PyroE peptide
  PyroE (1)..(1)
ETAVSSHAQD

SEQ ID NO: 31 - protein, 10 aa, Artificial Sequence
  PyroE peptide
  PyroE (1)..(1)
ETAVSSHEAD

SEQ ID NO: 32 - protein, 10 aa, Artificial Sequence
  PyroE peptide
  PyroE (1)..(1)
ETAVSSHEQA

SEQ ID NO: 33 - protein, 10 aa, Artificial Sequence
  PyroE peptide
  PyroE (1)..(1)
DTAVSSHEQD

SEQ ID NO: 34 - protein, 10 aa, Artificial Sequence
  PyroE peptide
  PyroE (1)..(1)
ESAVSSHEQD

SEQ ID NO: 35 - protein, 10 aa, Artificial Sequence
  PyroE peptide
  PyroE (1)..(1)
ETAISSHEQD

SEQ ID NO: 36 - protein, 10 aa, Artificial Sequence
  PyroE peptide
  PyroE (1)..(1)
ETALSSHEQD

SEQ ID NO: 37 - protein, 10 aa, Artificial Sequence
  PyroE peptide
  PyroE (1)..(1)
ETAMSSHEQD

SEQ ID NO: 38 - protein, 10 aa, Artificial Sequence
  PyroE peptide
  PyroE (1)..(1)
ETAVTSHEQD

SEQ ID NO: 39 - protein, 10 aa, Artificial Sequence
  PyroE peptide
  PyroE (1)..(1)
ETAVSTHEQD

SEQ ID NO: 40 - protein, 10 aa, Artificial Sequence
  PyroE peptide
  PyroE (1)..(1)
ETAVSSKEQD

SEQ ID NO: 41 - protein, 10 aa, Artificial Sequence
  PyroE peptide
  PyroE (1)..(1)
ETAVSSQEQD

SEQ ID NO: 42 - protein, 10 aa, Artificial Sequence
  PyroE peptide
  PyroE (1)..(1)
ETAVSSREQD

SEQ ID NO: 43 - protein, 10 aa, Artificial Sequence
  PyroE peptide
  PyroE (1)..(1)
ETAVSSHNQD

SEQ ID NO: 44 - protein, 10 aa, Artificial Sequence
  PyroE peptide
  PyroE (1)..(1)
ETAVSSHQQD

SEQ ID NO: 45 - protein, 10 aa, Artificial Sequence
  PyroE peptide
  PyroE (1)..(1)
ETAVSSHEDD

SEQ ID NO: 46 - protein, 10 aa, Artificial Sequence
  PyroE peptide
  PyroE (1)..(1)
ETAVSSHEND

SEQ ID NO: 47 - protein, 10 aa, Artificial Sequence
  PyroE peptide
  PyroE (1)..(1)
ETAVSSHEQN